(19) United States
(12) Patent Application Publication
(10) Pub. No.: US 2025/0195517 A1
(43) Pub. Date: Jun. 19, 2025

(54) TREATMENT OF INTRACRANIAL HEMORRHAGE

(71) Applicant: ImmunoChem Therapeutics, LLC, Newton, MA (US)

(72) Inventors: Linda J. VAN ELDIK, Lexington, KY (US); Martin D. WATTERSON, Chicago, IL (US); Saktimayee M. ROY, Evanston, IL (US)

(21) Appl. No.: 18/840,375

(22) PCT Filed: Feb. 21, 2023
(86) PCT No.: PCT/US2023/013526

§ 371 Date: Aug. 21, 2024

Related U.S. Application Data
(60) Provisional application No. 63/313,078, filed on Feb. 23, 2022.

(51) Int. Cl. A61K 31/506

(52) U.S. Cl. CPC A61K 31/506

(57) ABSTRACT

Described herein are compounds used to treat acute brain injury, pharmaceutical compositions and methods of use thereof.

### CROSS-REFERENCE

This application claims the benefit of U.S. Provisional Patent Application No. 63/313,078 filed on Feb. 23, 2022, entitled Treatment of Intracranial Hemorrhage, the entire contents of which are incorporated by reference herein.

### RESEARCH AND DEVELOPMENT

This invention was made with Government support under Grant No. R01 AG069930, awarded by the National Institutes of Health. The Government has certain rights in the invention.

### FIELD OF THE DISCLOSURE

The present invention is directed towards methods of treating nervous system injury and disease, and more particularly to compounds treating intracranial hemorrhage in humans, and medical uses thereof.

### BACKGROUND OF THE DISCLOSURE

Acute brain injuries resulting from trauma, such as traumatic brain injury (TBI) or non-traumatic cerebrovascular injury, such as intracranial hemorrhage, are major medical problems that cause considerable mortality and morbidity. In addition to damage from the initial insult, downstream pathophysiological events can result in further brain injury and lead to increased risk of longer-term neurologic complications. A key downstream injury response occurring within the relevant injury-to-treatment time window is a dysregulated inflammatory response in the brain (neuroinflammation). A specific aspect of neuroinflammation, injury-induced proinflammatory cytokine overproduction from abnormally activated glia, has been linked to subsequent neurological damage and cognitive deficits following acute brain injuries. In both animal models and human patients, acute brain injury induces a robust increase in cytokine levels in the brain that occurs in the first several hours to days after insult and then subsides. This acute proinflammatory cytokine surge is a key contributor to subsequent cerebral edema, long-term neuronal dysfunction, and cognitive impairment. This mechanistic linkage and the attractive therapeutic time window of hours to days post insult provides a rational therapeutic target for intervention in the acute care setting.

Injury-induced dysregulated glial activation is a critical convergence point for diverse central nervous system (CNS) insults that can compromise neuronal survival, and excessive glial activation is a major factor contributing to undesirable outcomes following acute brain injury. However, despite advances in our understanding of these cellular and molecular neuroinflammatory mechanisms underlying adverse neuronal sequelae following injury, approved therapeutics that target this pathological process are lacking. Thus, there is a clear and urgent need for a CNS-penetrant small-molecule drug candidate that improves neurological recovery and outcomes in acute brain injury conditions, particularly in humans.

### SUMMARY

Described herein are small molecule compounds that are CNS-penetrant small-molecules suitable for use in the treatment of acute brain injury, preferably in humans. Therefore, the compounds and compositions described herein can be used to treat acute brain injuries, such as, but not limited to, intracranial hemorrhage, such as intracerebral hemorrhage or subarachnoid hemorrhage.

In some embodiments described herein are pharmaceutical compositions comprising a compound as described herein, or a pharmaceutically acceptable salt or solvate thereof, and one or more pharmaceutically acceptable carriers, diluents and excipients suitable for administration to a patient in need of treatment for acute brain injury.

In some embodiments provided herein are methods for the treatment or prevention of the consequences of acute brain injury in a patient in need thereof, comprising administering to the patient a therapeutically acceptable dose of MW189, or a pharmaceutically acceptable salt or solvate thereof. In some embodiments provided herein are methods for the treatment or prevention of the consequences of acute brain injury in a patient in need thereof, comprising administering to the patient a pharmaceutical composition as described herein. In some embodiments, the acute brain injury is or comprises intracranial hemorrhage, including, but not limited to, epidural hematoma, subdural hematoma, subarachnoid hemorrhage, intracerebral hemorrhage, intraparenchymal hemorrhage, and intraventricular hemorrhage.

Other objects, features and advantages of the methods and compositions described herein will become apparent from the following detailed description. It should be understood, however, that the detailed description and the specific examples, while indicating specific embodiments, refers to such embodiments by way of illustration only, since various changes and modifications within the spirit and scope of the instant disclosure will become apparent to those skilled in the art from this detailed description.

### INCORPORATION BY REFERENCE

All publications, patents, and patent applications mentioned in this specification, if any, are herein incorporated by reference to the same extent as if each individual publication, patent, or patent application was specifically and individually indicated to be incorporated by reference.

### DETAILED DESCRIPTION

The compounds as described herein can be used in treating acute brain injury such as intracranial hemorrhage (e.g., hemorrhagic stroke) in a subject in need thereof. In some embodiments, the compounds as described herein prevent mortality or reduce the risk of mortality significantly in a subject in need thereof. In preferred embodiments, the subject is a human.

In some embodiments, a method for treating brain injury in a patient in need thereof includes administering to a patient suffering from acute brain injury an effective amount of one or more of the compounds or compositions disclosed herein. In some embodiments, the compounds or compositions as described herein reduce the risk of brain infection, e.g., by preventing or reducing the inflammatory responses to a systemic infection.

In preferred embodiments, the patient is human.

In some embodiments, the compounds as described herein can be used in treating acute brain injury such that the symptoms, consequences or sequelae of acute brain injury are significantly decreased. In some embodiments, cognitive impairment and memory problems associated with acute brain injury may be significantly reduced after treatment with an effective amount of the compounds disclosed herein. In some embodiments, the brain injury can be primary or secondary to an injurious event, such as hemorrhagic stroke. The primary injury may include intracranial hematomas, including, but not limited to, epidural hematoma, subdural hematoma, subarachnoid hemorrhage, intracerebral hemorrhage, intraparenchymal hemorrhage, and intraventricular hemorrhage.

In some embodiments, the secondary brain injury may include hypoxia and hypotension, ischemia, cerebral edema, hydrocephalus, hemorrhage expansion, or second impact syndrome. In some embodiments, secondary brain injury may also manifest as cognitive and behavioural problems.

In some embodiments, the compounds described herein prevent or reduce the severity of secondary brain injury after the initial insult, in a subject in need thereof.

The compounds as described herein can be used in treating traumatic brain injury in a subject, in need thereof.

The compounds as described herein can also be used in treating spinal cord injury in a subject, in need thereof.

In some embodiments, the compounds described herein regenerate or aid in the regeneration of cells in a subject in need thereof. In embodiments, the compounds described herein regenerate or aid in the regeneration of brain cells, in a subject in need thereof.

In some embodiments, the method of treating brain injury or intracranial hemorrhage may include selective suppression of injury and/or disease-induced glial proinflammatory cytokine overproduction associated with destructive glial inflammation/synaptic dysfunction cycles. In some embodiments, treating the acute brain injury includes reducing perihematomal edema during and after treatment.

In some embodiments, the method of treating brain injury or intracranial hemorrhage includes reducing one or more pro-inflammatory cytokines or chemokines, including, but not limited to, interleukin-1 (IL-1)β, IL-2, IL-6, IL-8, L-9, IL-12, IL-15, IL-17, IL-18, IL-33, MCP-1 (CCL2), IP-10 (CXCL10), tumor necrosis factor alpha (TNF-α), MIP-1α, MIP-2, interferon gamma (IFNγ), KC/GRO, or granulocyte-macrophage colony stimulating factor (GM-CSF). In some embodiments, one or more pro-inflammatory cytokines may be used as biomarkers, which may be detected and/or quantitated to diagnose acute brain injury, such as intracranial hemorrhage, to assess effectiveness of therapy, or both.

In some embodiments, the method of treating acute brain injury or intracranial hemorrhage may include increasing one or more anti-inflammatory cytokines, including interleukin (IL)-1 receptor antagonist, IL-4, IL-5, IL-10, IL-11, IL-13, or IL-35. In some embodiments, one or more anti-inflammatory cytokines may be used as biomarkers, which may be detected and/or quantitated to diagnose acute brain injury, such as intracranial hemorrhage, to assess effectiveness of therapy, or both.

In some embodiments, basal physiological cytokine levels do not change with the administration of one or more compounds disclosed herein at efficacious dose levels.

In some embodiments, the compounds disclosed herein can be used in combination with ion channel modulators. In some embodiments, the compounds disclosed herein can be used in combination with nimodipine to treat acute brain injury.

In some embodiments, the compounds disclosed herein can be used in combination with other therapeutic agents such as anti-hypertensive agents, diuretics, anti-pyretics, analgesics, or anticonvulsants.

In some embodiments, the method of treating brain injury or intracranial hemorrhage includes reduction in neurofilament light chain polypeptide (“NfL” or “neurofilament light chain”).

In some embodiments, compounds of this disclosure or a pharmaceutically acceptable salt or solvate thereof, may be administered in an amount (measured in milligrams (mg)) proportional to a patient's body weight in kilograms (kg). Such amounts may be expressed in mg/kg or mpk. In some embodiments, the compounds described herein may be administered in an amount of at least about 0.01, 0.025 mpk, 0.05 mpk, 0.2 mpk, 0.3 mpk, 0.4 mpk, 0.5 mpk, 1 mpk, 2 mpk, 4 mpk, 5 mpk, 10 mpk, 15 mpk, 20 mpk, or 30 mpk. In some preferred embodiments, the compounds described herein may be administered in an amount of about 0.25 mpk, about 0.5 mpk, about 0.75 mpk, about 1 mpk, about 1.5 mpk, about 2 mpk, or about 2.5 mpk.

In some embodiments, the compounds of this disclosure or a pharmaceutically acceptable salt or solvate thereof, are administered in an effective amount from of about 0.01 to about 1 mpk. Preferably, the effective amount is from of about 0.1 to about 0.5 mg per kg of the patient's body weight (mpk). More preferably, the effective amount is from of about 0.15 to about 0.3 mg per kg of the patient's body weight (mpk).

In some embodiments, the compounds of this disclosure or a pharmaceutically acceptable salt or solvate thereof, are administered to a human subject with a brain hemorrhage that is between 10 mL and 60 mL in volume, as confirmed via diagnostic and stability computerized tomography (CT) scans utilizing volumetric assessment, within 24 hours of initial insult or symptom onset and approximately every 12 hours for up to 5 days (10 total doses) thereafter or until the human subject is discharged from the hospital (if earlier than 5 days). In some embodiments the brain hemorrhage may be between 1 mL and 100 mL in volume.

In some embodiments, the compounds of this disclosure or a pharmaceutically acceptable salt or solvate thereof, are administered from about 1 to 4 times per day. In some embodiments, the compounds of this disclosure or a pharmaceutically acceptable salt or solvate thereof, are administered from about once or twice a day. In some preferred embodiments, the compounds of this disclosure or a pharmaceutically acceptable salt or solvate thereof, are administered twice per day.

In some embodiments, the compounds of this disclosure, or a pharmaceutically acceptable salt, or solvate thereof, can be administered in a dosing regimen. The regimen can be structured to provide therapeutically effective amounts of the compounds over a predetermined period of time (e.g., an administration time). The regimen can be structured to limit or prevent side-effects or undesired complications of the compounds disclosed herein. Regimens useful for treating acute brain injury can include any number of days of administration which can be repeated as necessary. Administration periods can be broken by a rest period that includes no administration. For example, a regimen can include administration periods that include 2, 3, 5, 7, 10, 15, 21, 28, or more days. These periods can be repeated. For example, a regimen can include a set number of days as previously described where the regimen is repeated 1, 2, 3, 4, 5, 6, 7, 8, 9, 10, 11, 12, 13, or more times.

In some embodiments, the administration of the compounds of this disclosure can be delayed until up to about 4 to 8 hours after injury. In some embodiments, the administration of the compounds of this disclosure, or a pharmaceutically acceptable salt, or solvate thereof, can be delayed until up to about 10 hours after injury. In preferred embodiments, the administration of the compounds of this disclosure (e.g., MW189), or a pharmaceutically acceptable salt, or solvate thereof, can be delayed up to about 24 hours after injury. In some embodiments, administration of the compounds of this disclosure may be administered up to about 8, 16, 24, 36 or 48 hours after injury.

In some embodiments, the administration of the compounds of this disclosure commences within from about 1 minute to 24 hours, about 10 min. to about 12 hours, or about 30 min. to about 6 hours after the patient suffers an acute brain injury. In some embodiments, the administration of the compounds of this disclosure commences within from about between 4-6 hours, or about between 6-12 hours, or about between 12-24 hours, or about between 24-36 hours.

In some embodiments, the administration of the compounds of this disclosure commences within from about 2-36 hours, about 4-36 hours, about 6-36 hours, about 6-18 hours, about 6-8 hours or about 2-24 hours, about 4-12 hours, about 4-24 hours, 4-36 hours, about 6-12 hours, about 6-24 hours, about 6-36 hours, about 8-12 hours, about 8-24 hours, about 8-36 hours, about 10-12 hours, about 10-18 hours, about 10-24 hours, about 10-36 hours, about 12-24 hours, about 12-36 hours, about 18-24 hours, or about 18-36 hours after the patient suffers an acute brain injury.

In some embodiments, the typical daily dose of the compounds of this disclosure or pharmaceutically acceptable salts thereof, varies within a wide range and will depend on various factors such as the relevant indication, severity of the illness being treated, the route of administration, the age, weight and sex of the patient and the particular compound being used, and can be determined by a physician.

In some embodiments, the compounds described herein are formulated into pharmaceutical compositions. In some embodiments, the pharmaceutical composition comprising the compounds described herein is administered to a subject in a variety of ways by multiple administration routes, including, but not limited to, oral, parenteral (e.g., intravenous, subcutaneous, intramuscular, intramedullary injections, intrathecal, direct intraventricular, intraperitoneal, or intralymphatic injections), intranasal, buccal, topical or transdermal administration routes. In some embodiments, the pharmaceutical composition comprising the compounds described herein is administered to a subject parenterally. In some preferred embodiments, the pharmaceutical composition comprising the compounds described herein is administered to a subject intravenously.

In some embodiments, the compounds described herein are formulated into pharmaceutical composition capable of passing through the blood brain barrier.

In some embodiments, the compounds of this disclosure include MW01-6-189WH (MW189):

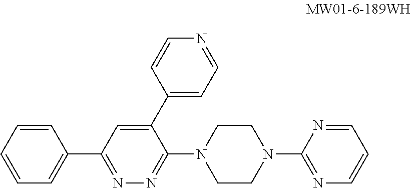 [SMILES: c1ccc(-c2cc(-c3ccncc3)c(N3CCN(c4ncccn4)CC3)nn2)cc1]

### (MW189)

In some embodiments, compounds described herein include MW189 and pharmaceutically acceptable salts, solvates, polymorphs or prodrugs thereof.

In some preferred embodiments, MW189 may be administered as part of a method for treating acute brain injury in a patient in need thereof. The method includes administering to a patient suffering from acute brain injury an effective amount of a compound as described herein, such as MW189.

In some embodiments, the acute brain injury comprises intracranial hemorrhage, including, but not limited to, epidural hematoma, subdural hematoma, subarachnoid hemorrhage, intracerebral hemorrhage, intraparenchymal hemorrhage, and intraventricular hemorrhage.

### Pharmaceutical Compositions

In some embodiments, the compounds described herein are formulated into pharmaceutical compositions. Pharmaceutical compositions may be formulated in a conventional manner using one or more pharmaceutically acceptable inactive ingredients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

A pharmaceutical composition, as used herein, refers to a mixture of a compound disclosed herein with other chemical components (i.e., pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. The pharmaceutical composition facilitates administration of the compound to an organism, including a human subject.

Pharmaceutical formulations described herein are administrable to a subject in a variety of ways by multiple administration routes, including, but not limited to, oral, parenteral (e.g., intravenous, subcutaneous, intramuscular, intramedullary injections, intrathecal, direct intraventricular, intraperitoneal, intralymphatic injections), intranasal, buccal, topical or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

In some embodiments, the compounds disclosed herein may be administered orally.

In some embodiments, the compounds disclosed herein are administered topically. In such embodiments, the compounds disclosed herein may be formulated into a variety of topically administrable compositions, such as solutions, suspensions, lotions, gels, pastes, shampoos, scrubs, rubs, smears, medicated sticks, medicated bandages, balms, creams or ointments. In some embodiments, the compounds disclosed herein may be administered topically to the skin.

In another aspect, the compounds disclosed herein may be administered by inhalation.

In another aspect, the compounds disclosed herein are formulated for intranasal administration. Such formulations include nasal sprays, nasal mists, and the like.

In another aspect, the compounds disclosed herein are formulated as eye drops.

In any of the aforementioned aspects are further embodiments in which the effective amount of a compound disclosed herein (e.g., MW189) is: (a) systemically administered to the mammal; and/or (b) administered orally to the mammal; and/or (c) intravenously administered to the mammal; and/or (d) administered by inhalation to the mammal; and/or (e) administered by nasal administration to the mammal; or and/or (f) administered by injection to the mammal; and/or (g) administered topically to the mammal; and/or (h) administered by ophthalmic administration; and/or (i) administered rectally to the mammal; and/or (j) administered non-systemically or locally to the mammal.

In any of the aforementioned aspects are further embodiments comprising single administrations of the effective amount of a compound disclosed herein (e.g., MW189), including further embodiments in which (i) the compound is administered once; (ii) the compound is administered to the mammal multiple times over the span of one day; (iii) the compound is administered continuously over a period of time, such as 1-10 days, 1-10 weeks, 1-20 weeks, 1-30 weeks, etc.; or (iv) the compound is administered continually over an indefinite period of time.

In any of the aforementioned aspects are further embodiments comprising multiple administrations of the effective amount of a compound disclosed herein (e.g., MW189), including further embodiments in which (i) the compound is administered continuously or intermittently: as in a single dose; (ii) the time between multiple administrations is every 6 hours; (iii) the compound is administered to the mammal every 8 hours; (iv) the compound is administered to the mammal every 12 hours; (v) the compound is administered to the mammal every 24 hours. In further or alternative embodiments, the method comprises a drug holiday, wherein the administration of the compound disclosed herein is temporarily suspended or the dose of the compound being administered is temporarily reduced; at the end of the drug holiday, dosing of the compound is resumed. In some embodiments, the length of the drug holiday varies from 2 days to 1 year.

In certain embodiments, the compounds disclosed herein are administered in a local rather than systemic manner.

In some embodiments, a compound disclosed herein may be administered topically.

In some embodiments, a compound disclosed herein may be administered systemically.

In some embodiments, a pharmaceutical formulation may be in the form of a tablet or a capsule.

In some embodiments, liquid formulation dosage forms for oral administration are in the form of aqueous suspensions or solutions selected from the group, including, but not limited to, aqueous oral dispersions, emulsions, solutions, elixirs, gels, and syrups.

For administration by inhalation, a compound disclosed herein (e.g., MW189) may be formulated for use as an aerosol, a mist or a powder.

For buccal or sublingual administration, compositions comprising compounds disclosed herein (e.g., MW189) may take the form of tablets, lozenges, or gels formulated in a conventional manner.

In some embodiments, compounds disclosed herein may be prepared as transdermal dosage forms.

In some embodiments, a compound disclosed herein may be formulated into a pharmaceutical composition suitable for intramuscular, subcutaneous, or intravenous injection.

In some embodiments, a compound disclosed herein may be administered topically and can be formulated into a variety of topically administrable compositions, such as solutions, suspensions, lotions, gels, pastes, medicated sticks, balms, creams or ointments.

In some embodiments, the compounds disclosed herein are formulated in rectal compositions such as enemas, rectal gels, rectal foams, rectal aerosols, suppositories, jelly suppositories, or retention enemas.

### Methods of Dosing and Treatment Regimens

In some embodiments, the compounds disclosed herein are used in the preparation of medicaments for the treatment of diseases or conditions described herein. In addition, a method for treating any of the diseases or conditions described herein in a subject in need of such treatment, involves administration of pharmaceutical compositions that include at least one compound disclosed herein or a pharmaceutically acceptable salt, active metabolite, prodrug, or solvate thereof, in therapeutically effective amounts to said subject.

In certain embodiments, the compositions containing a compound disclosed herein (e.g., MW189) are administered for prophylactic and/or therapeutic treatments. In certain therapeutic applications, the compositions may be administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts may optionally be determined by methods, including, but not limited to, a dose escalation clinical trial.

In prophylactic applications, compositions containing the compounds disclosed herein are administered to a patient susceptible to or otherwise at risk of a particular disease, disorder or condition.

In certain embodiments, the dose of drug being administered may be temporarily reduced or temporarily suspended for a certain length of time (i.e., a “drug holiday”).

Doses employed for adult human treatment are typically in the range of 0.05 mpk to 2.5 mpk, which may be administered from one to six times daily.

### Methods of Treatment

Described herein are methods for the treatment of acute brain injury, including intracranial hemorrhage, such as intracerebral hemorrhage or subarachnoid hemorrhage. In some embodiments, described herein is a method of treating a disease or condition, including administering to a subject in need thereof an effective amount of a compound disclosed herein.

### Definitions

As used in this specification and the appended claims, the singular forms “a,” “an,” and “the” include plural referents unless the content clearly dictates otherwise. It should also be noted that the term “or” is generally employed in its sense, including “and/or,” unless the content clearly dictates otherwise. Further, headings provided herein are for convenience only and do not interpret the scope or meaning of the claimed invention.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms and/or consequences. An appropriate “effective” amount in any individual case may be determined using techniques, such as a dose escalation study. An “effective amount” is an amount sufficient for a compound to accomplish a stated purpose relative to the absence of the compound (e.g., achieve the effect for which it is administered, treat a disease, reduce enzyme activity, increase enzyme activity, reduce a signaling pathway, or reduce one or more symptoms and/or consequences of a disease or condition, reduce or increase one or more biomarkers indicative of occurrence or severity of a disease or condition, or a combination of two or more thereof). An example of an “effective amount” is an amount sufficient to contribute to the treatment, prevention, or reduction of a symptom or symptoms of a disease, which could also be referred to as a “therapeutically effective amount.”

A “reduction” of a symptom or symptoms (and grammatical equivalents of this phrase) means decreasing of the severity or frequency of the symptom(s), or elimination of the symptom(s).

A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. A prophylactic effect may be whole or partial. An “activity decreasing amount,” as used herein, refers to an amount of antagonist required to decrease the activity of an enzyme relative to the absence of the antagonist.

The exact amounts corresponding with an “effective amount” or “therapeutically effective amount” will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “brain injury” refers to a condition in which the brain is damaged by injury caused by an event. As used herein, an “injury” is an alteration in cellular or molecular integrity, activity, level, robustness, state, or other alteration that is traceable to an insult that causes the injury. For example, an injury includes a physical, mechanical, chemical, biological, functional, infectious, or other modulator of cellular or molecular characteristics. An insult can include a physical trauma such as a single or repetitive impact (percussive) or a biological abnormality such as a stroke resulting from either blockade or leakage of a blood vessel. An insult may be, or include, an infection by an infectious agent. A person of skill in the art will recognize numerous events that are encompassed by the terms injury or insult.

More specifically, the term “brain injury” refers to a condition that results in central nervous system damage, irrespective of its pathophysiological basis. Stroke is among the most frequent origins of a “brain injury.” A “stroke” may be classified into hemorrhagic and non-hemorrhagic strokes. Examples of hemorrhagic stroke include cerebral hemorrhage, subarachnoid hemorrhage, and intracranial hemorrhage secondary to cerebral arterial malformation, while examples of non-hemorrhagic stroke include cerebral infarction.

The term “subject” or “patient” encompasses organisms, including mammals. Examples of mammals include, but are not limited to, humans. In some preferred embodiments described herein, the mammal is a human.

The terms “treat,” “treating” or “treatment,” as used herein, include alleviating, abating or ameliorating one or more symptoms, consequence, results, and/or sequelae of a disease or condition, preventing additional symptoms, inhibiting the disease or condition, e.g., arresting the development of the disease or condition, relieving the disease or condition, causing regression of the disease or condition, relieving a condition caused by the disease or condition, preventing the development or progression of a condition caused by the disease or condition, or stopping the symptoms of the disease or condition either prophylactically and/or therapeutically. Efficacy of treatment of brain injury may be evaluated by various methods, which may comprise detecting and/or quantitating one or more biomarkers in one or more tissues and/or fluids, such as the blood, blood serum, cerebrospinal fluid (CSF), etc. Biomarker levels may be used to diagnose and/or assess the severity of acute brain injury, e.g., by determining an amount of biomarker in a biological fluid, such as blood plasma or CSF, and comparing such amount to a normal quantity of such biomarker in tissue and/or biological fluid from one or more uninjured subjects of the same species of patient (e.g., human). Such biomarkers may comprise, e.g., neurofilament light chain polypeptide (NfL), which may be detected in blood serum and/or CSF. Such biomarkers may also comprise one or more pro-inflammatory cytokines, as described herein.

### EXAMPLE

The compounds disclosed herein can be used to treat acute brain injury, preferably in a human patient.

MW189 is a novel small molecule drug candidate developed as a selective suppressor of disease- and injury-induced proinflammatory cytokine overproduction associated with destructive neuroinflammation/synaptic dysfunction cycles. In animal models of acute brain injuries such as intracranial hemorrhage, MW189 attenuates neuroinflammation, reduces cerebral edema, and improves functional and cognitive performance. The investigators seek to establish if these targets are modified in humans with intracranial hemorrhage.

Experimental method: To determine the safety, tolerability, biological response and pharmacokinetic outcomes of MW189 in treatment of acute brain injury, MW189 is administered intravenously to human subjects suffering from intracranial hemorrhage. In this study, MW189 (0.25 mg/kg) is administered to human subjects with a brain hemorrhage that is between 10 mL and 60 mL in size, as confirmed via diagnostic and stability CT scans utilizing volumetric assessment, within 24 hours of initial insult or symptom onset and approximately every 12 hours for up to 5 days (10 total doses) thereafter or until the human subject is discharged from the hospital (if earlier than 5 days).

Results: Biochemical biomarkers, such as NfL and cytokines, are used to demonstrate target engagement and biological response. Safety and tolerability of MW189 are evaluated, e.g., by monitoring patients' health and pharmacokinetic parameters and/or one or more biomarkers. Exploratory radiographic and clinical endpoints are also monitored. The production of proinflammatory cytokines is also monitored. Secondary brain tissue damage resulting from intracranial hemorrhage is also monitored.

The examples and embodiments described herein are intended for illustrative purposes only and various modifications or changes to the specific embodiments described herein will be apparent to persons skilled in the art and are to be included within the spirit and purview of this application and scope of the appended claims. All publications, patents, and patent applications cited herein are incorporated by reference in their entirety for all purposes.

1. A method of treating acute brain injury in a patient in need thereof, comprising administering to a patient suffering from acute brain injury an effective amount of MW189.

2. The method of claim 1, further comprising detecting one or more biomarkers to diagnose or assess the severity of the acute brain injury, assess response of the patient to MW189, or combinations thereof.

3. The method of claim 2, wherein the biomarker level is indicative of the occurrence, severity, or both, of the acute brain injury and a change in one or more biomarker levels is indicative of the patient's response to MW189.

4. The method of claim 1, comprising
a. detecting one or more biomarkers to diagnose and assess the severity of acute brain injury prior to administration of MW189;
b. administering MW189;
c. at a time after administration of MW189, detecting the one or more biomarkers to assess a change in the severity of the acute brain injury; and
d. continuing, discontinuing, increasing, or decreasing administration of MW189.

5. The method of claim 1, wherein the biomarker comprises neurofilament light chain.

6. The method of claim 1, wherein the acute brain injury is hemorrhagic stroke.

7. The method of claim 1, wherein the patient is a human.

8. The method of claim 1, wherein said administration commences within from about between 4-6 hours, about between 6-12 hours, about between 12-24 hours, or about between 24-36 hours after the patient suffers an acute brain injury.

9. The method of claim 1, wherein treating acute brain injury or intracranial hemorrhage comprises preventing morbidity and/or mortality or reducing a risk of morbidity and/or mortality.

10. The method of claim 1, wherein treating brain injury or intracranial hemorrhage comprises reducing the risk of morbidity and/or mortality of the patient.

11. The method of claim 1, wherein treating brain injury or intracranial hemorrhage comprises reducing one or more pro-inflammatory cytokines.

12. The method of claim 11, wherein said one or more pro-inflammatory cytokines comprise TNF-α.

13. The method of claim 1, wherein treating brain injury or intracranial hemorrhage comprises increasing one or more anti-inflammatory cytokines.

14. The method of claim 13, wherein said one or more anti-inflammatory cytokines comprises IL-10.

15. The method of claim 1, wherein the effective amount of MW189 is about 0.025 to 30 mg/kg.

16. The method of claim 15, wherein the effective amount of MW189 is about 0.25 mg/kg.

17. The method of claim 1, comprising administering the MW189 from one to four times per day.

18. The method of claim 17, wherein MW189 is administered once or twice per day.

19. The method of claim 18, wherein MW189 is administered twice per day.

20. The method of claim 1, wherein administering MW189 to the patient comprises administering MW189 to the patient on at least two consecutive days.

21. The method of claim 20, comprising administering MW189 to the patient on two, three, four, five, six, seven, eight, nine, ten or more consecutive days.

22. The method of claim 1, wherein treating acute brain injury comprises preventing brain infection or reducing a risk of brain infection.

23. The method of claim 1, wherein treating acute brain injury comprises reducing intracranial hemorrhage, preventing or reducing a risk of hemorrhage expansion, or both.

24. The method of claim 1, wherein treating acute brain injury comprises reducing perihematomal edema during and after treatment.

25. The method of claim 1, wherein treating acute brain injury comprises reducing secondary brain tissue damage consequential to or resulting from the acute brain injury.

26. The method of claim 1, comprising administering to a patient suffering from acute brain injury an effective amount of MW189 in combination with a therapeutic agent.

27. The method of claim 1, comprising administering to a patient suffering from acute brain injury an effective amount of MW189 in combination with an ion channel modulator.

28. The method of claim 1, comprising administering to a patient suffering from acute brain injury an effective amount of MW189 in combination with an ion channel modulator and a therapeutic agent.

29. The method of claim 26, wherein the therapeutic agent comprises an anti-hypertensive, a diuretic, an anti-pyretic, an analgesic, or an anticonvulsant.

30. The method of claim 27, wherein the ion channel modulator comprises nimodipine.
